(19) United States
(12) Patent Application Publication
(10) Pub. No.: US 2008/0058940 A1
(43) Pub. Date: Mar. 6, 2008

(54) Artificial intervertebral disc

(76) Inventors: Shing Sheng Wu, Taipei City (TW); Po-Quang Chen, Taipei City (TW); Hon So, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/507,588

(22) Filed: Aug. 22, 2006

(51) Int. Cl. A61F 2/44

(52) U.S. Cl. 623/17.15

(57) ABSTRACT

An intervertebral disc according to this invention comprises an upper lid, a lower lid, and a joint piece, and the upper lid is formed with a first sphere and a first shaft and the lower lid is formed with a first camber and a second shaft, in which the top of joint piece is formed with a second sphere and a first guidance groove and the bottom of joint piece is formed with a second camber and a second guidance groove; the joint piece is provided between the upper lid and the lower lid, makes the second sphere and the second camber glide and roll respectively on the first sphere and the first camber to contact with each other, and makes the first shaft and the second shaft respectively fit to the first guidance groove and the second guidance groove, thereby the intervertebral disc adjustable being formed.

### BACKGROUND OF THE INVENTION

1. Field of the Invention

This invention relates to an artificial intervertebral disc and particularly to an artificially made intervertebral disc that may be permanently planted between two joints of vertebrae of a human body.

2. Description of Related Art

A conventional artificial intervertebral disc is implanted between two joints of vertebrae of a human body to substitute for a cartilage tissue that degenerates and deforms. For example, in Taiwan Patent No. 360045, titled “INTERVERTEBRAL DISC SUBSTITUTE (1)”, the substitute is a slightly cuneiform structure of which one end is wide and the other one is narrow, and wedge teeth are provided at two outsides of the substitute, respectively; when the substitute is implanted between the two joints of vertebrae from the back, the wide end is at the side of belly and the narrow side is at the side of back, and further the wedge teeth of two sides are wedged to the surface of an upper vertebra and the surface of a lower vertebra.

Further, in Taiwan Patent No. 395237, titled “INTERVERTEBRAL DISC SUBSTITUTE”, the substitute is structured with two support plates and an adjustable flake. Two ends of each support plate are respectively a belly end and a back end, and a lug and a groove are provided at the back end of each support plate. And an assembly groove is provided at the belly end of each support plate, and an assembly lug is provided at two sides of the adjustable flake, fitting to the assembly groove; thereby, the lug may be assembled with the groove between the two support plates, and the adjustable flake is installed between the assembly groove of the each support plate to form a 3D structure.

However, the conventional intervertebral disc is designed in a constant way and cannot be properly adjusted on an effective demand when implanted between the two joints of vertebrae of the human body, so that the purpose of intervertebral disc is limited.

For this reason, in consideration of improvability of the defects described above, this inventor especially concentrates on studies and operate in coordination with academic theories, finally providing this invention for a design reasonable and effective improvement of the defects mentioned above.

### SUMMARY OF THE INVENTION

It is an object of this invention to mainly provide an intervertebral disc. However, the intervertebral disc is designed in an adjustable way and may be properly adjusted on an effective demand when implanted between two joints of vertebrae of a human body, so that the purpose of intervertebral disc is preferable.

In order to achieve the objective mentioned above, the intervertebral disc according to this invention comprises an upper lid of which the bottom is concave provided with a first space inside which a first sphere is formed inside which a shaft is provided; a lower lid of which the top is concave provided with a second space inside which a camber is formed inside which a second shaft is provided; a joint piece of which the top and the bottom are respectively formed with a second sphere and a second camber that are corresponding to the first sphere and the first camber, in which the second sphere and the second camber are respectively provided with a first guidance groove and a second guidance groove; the joint piece is provided between the upper lid and the lower lid and make the second sphere and the second camber glide and roll on the first sphere and the camber to contact with each other, and make the first shaft and the second shaft glide respectively fit to the first guidance groove and the second guidance groove.

In order to further know the features and technical means of this invention, refer to the detailed description according to this invention accompanied with drawings; however, the accompanied drawings are provided for reference and illustration only and are not limited to this invention.

### BRIEF DESCRIPTION OF THE DRAWINGS

FIG. 1 is a 3D exploded view of an intervertebral disc according to this invention;

FIG. 2 is a 3D assembly view of the intervertebral disc according to this invention;

FIG. 3 is a 3D view of an upper lid of the intervertebral disc according to this invention;

FIG. 4 is a 3D view of joint pieces of the intervertebral disc according to this invention; and

FIG. 5 is a schematic view illustrating the intervertebral disc according to this invention that is implanted between two joints of vertebrae.

### DETAILED DESCRIPTION OF THE PREFERRED EMBODIMENTS

Now, the present invention will be described more specifically with reference to the following embodiments. It is to be noted that the following descriptions of preferred embodiments of this invention are presented herein for purpose of illustration and description only; it is not intended to be exhaustive or to be limited to the precise form disclosed.

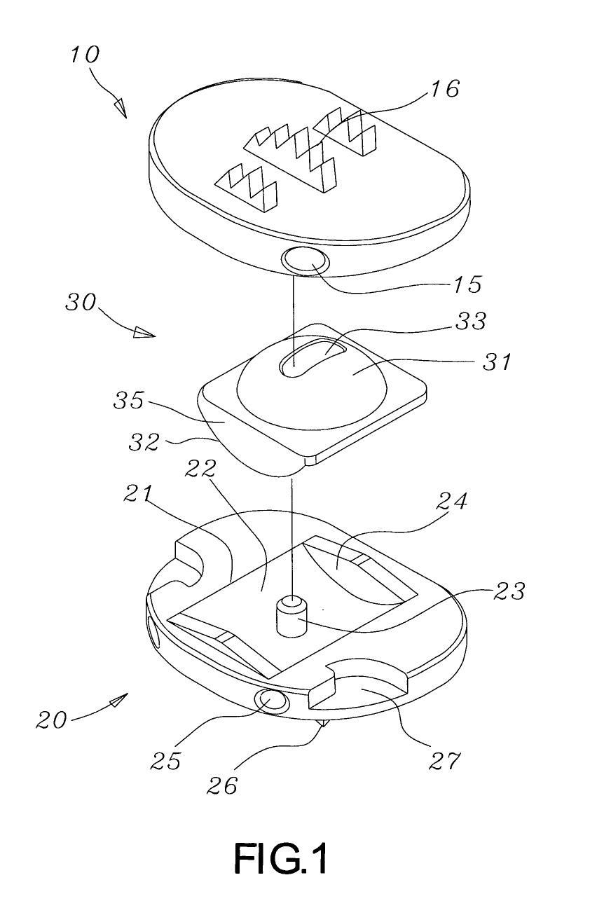

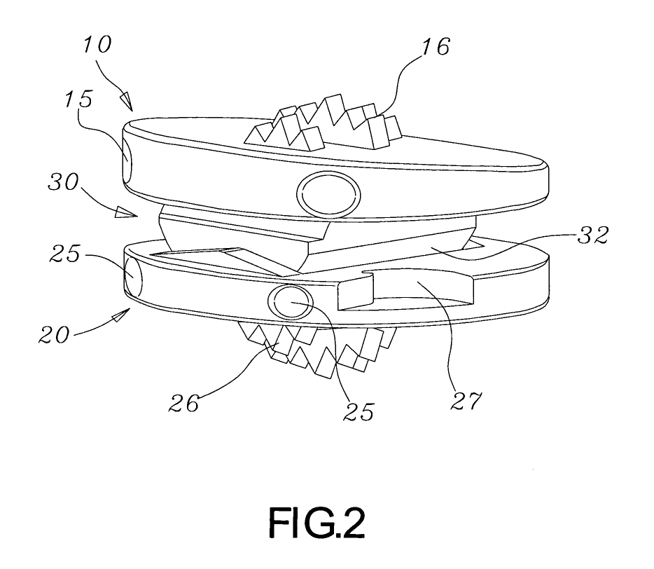

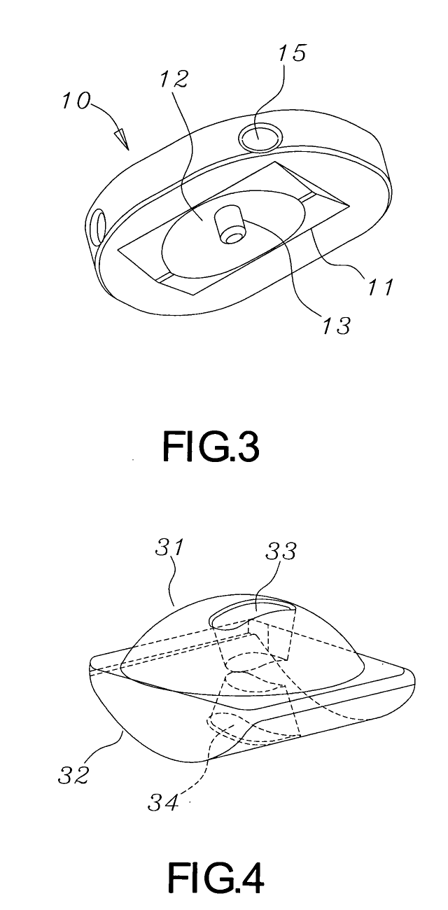

As shown in FIGS. 1 and 2, the intervertebral disc according to this invention comprises an upper lid 10, a lower lid 20, and a pair of joint pieces 30 and the materials of the members are not limited, in which the upper lid 10 is a flat plate of which the bottom is concave provided with a first space 11, as shown in FIG. 3, inside which a first sphere 12 is concave formed inside which the center is formed with a round first shaft 13, the exterior of the upper lid 10 is formed with two pairs of fitting hole 15, and the top of the upper lid 10 is formed with a plurality of wedge teeth 16 that may be used to wedge to the surface of an upper vertebra.

The lower lid 20 is located below the upper lid 10 and is formed into a flat plate mostly corresponding to the upper lid 10. The top of the lower lid 20 is concave provided with a second space 21 inside which a first camber 22 is formed inside which a round second shaft 23 is provided. First blocking sides 24 are formed at two sides corresponding to each other in the second space 21. Further, two pairs of fitting holes 25 and two hooked clipping grooves 27 are provided at the exterior of the lower lid 20 of which the bottom is formed with a plurality of wedge teeth 26 that may be used to wedge to the surface of an upper vertebra.

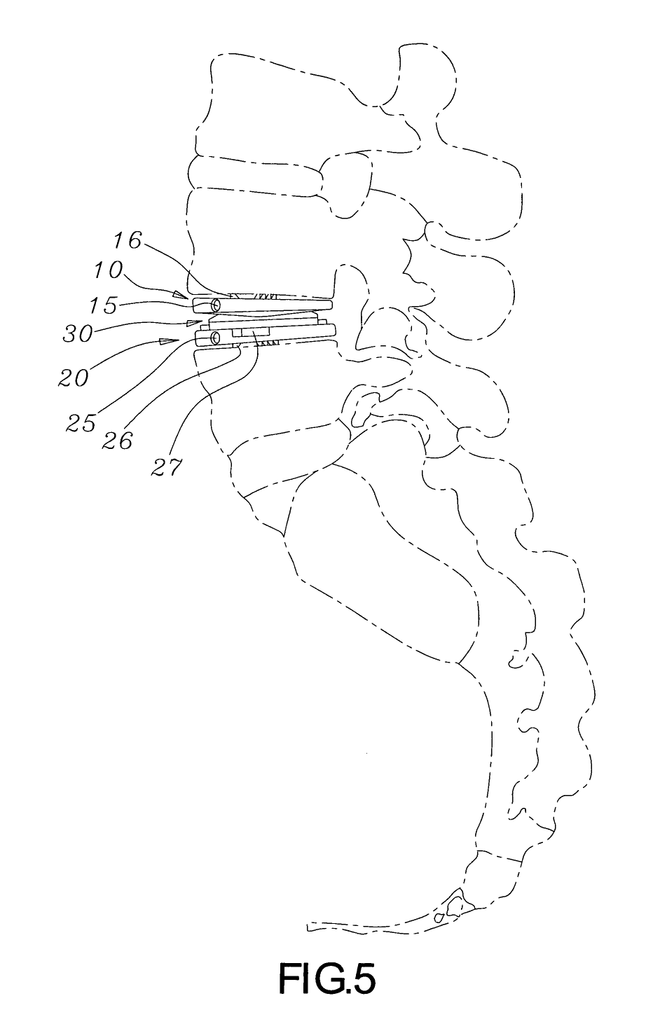

The top and bottom of the joint piece 30 are formed respectively with a second sphere 31 and a second camber 32, in which the second sphere 31 is convex corresponding to the first sphere 12, the second sphere 31 and the second camber 32 are corresponding respectively to the first sphere 12 and the first camber 22, and a first guidance groove 33 and a second guidance groove 34 are formed respectively in the center on the second sphere 31 and the second camber 32, as shown in FIG. 4, in which the first guidance groove 33 and the second guidance groove 34 vertically extend respectively for a length in the direction of the second sphere 31 and the second camber 32, and second blocking sides 35 are formed at two sides of the joint piece 30 that are corresponding to each other.

The joint piece 30 is provided between the upper lid 10 and the lower lid 20, the second sphere 31 and the second camber 32 of the joint piece 30 are made to glide and roll respectively on the first sphere 12 of the upper lid 10 and the first camber 22 of the lower lid 20 to contact with each other, and the first shaft 13 of the upper lid 10 and the second shaft 23 of the lower lid 20 are respectively inserted to glide correspondingly in the first guidance groove 33 and the second groove 34 of the joint piece 30 of which the second blocking side 35 contacts the first blocking side 24 of the lower lid 20.

Thus, the match of first sphere 12 with second sphere 31 and the match of first shaft 13 and guidance groove 33 may make the upper lid 10 and the joint piece 30 turn around each other frontward and backward or leftward and rightward. The match of first camber 22 and second camber 32 and the match of second shaft and second guidance groove 34 may make the lower lid 20 and the joint piece 30 turn around each other leftward and rightward, thereby the intervertebral disc according to this invention being formed.

The intervertebral disc according to this invention may be implanted between the two joints of vertebrae and the wedge teeth 16 and 26 above and below the intervertebral disc may be used to respectively wedge to the surfaces of upper and lower vertebrae so as to make the intervertebral disc closely occlude the vertebrae, as shown in FIG. 5.

The upper lid 10, the lower lid 20, and the joint piece 30 are designed in a movable way so that the intervertebral disc may be designed in an adjustable way, implanted between two joints of vertebrae of the human body, and properly adjusted on an effective demand, thereby the purpose of intervertebral disc being preferable. Especially, the match of first sphere 12 and second sphere 31 makes the upper lid 10 and the joint piece 30 turn around each other at the angle of 360 leftward and rightward so that the upper lid 10 and the joint piece 30 may be flexibly adjusted, thereby the purpose of intervertebral disc being preferable.

To sum up, this invention, a new type of product hard to come by, available, novel, and progressive in the industry, completely meet the requirements of application for the new type patent, and hence we apply following the patent law; we earnestly request you to examine it for details and to approve the patent as soon as possible for protection of the inventor's rights and interests.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. An intervertebral disc, comprising:
an upper lid provided with a first space, a first sphere formed in the first space, a first shaft provided on the first sphere;
a lower lid provided with a second space, a first camber is formed in the second space, a second shaft provided in the first chamber;
a joint piece comprising a second sphere and a second camber respectively corresponding to the first sphere and the first camber, the second sphere and the second camber respectively provided with a first guidance groove and a second guidance groove;
wherein the joint piece is provided between the upper lid and the lower lid and makes the second sphere and the second camber glide and roll respectively on the first sphere and in the first camber to contact with each other, and makes the first shaft and the second shaft glide respectively and fit the first guidance groove and the second guidance groove.

2. The intervertebral disc according to claim 1, wherein the first sphere is concave and the second sphere is convex corresponding to the first sphere.

3. The intervertebral disc according to claim 1, wherein the first guidance groove and the second groove extend vertically.

4. The intervertebral disc according to claim 1, wherein the exterior of upper lid and that of lower lid are respectively formed with a fitting hole.

5. The intervertebral disc according to claim 1, wherein the top of upper lid and the bottom of lower lid are respectively formed with a plurality of wedge teeth.

6. The intervertebral disc according to claim 1, wherein first blocking sides are formed at two sides corresponding to each other in the second space of the lower lid, second blocking sides are formed at two sides of the joint piece that are corresponding to each other, and the second blocking sides contact the first blocking sides.

7. The intervertebral disc according to claim 1, wherein the exterior of the lower lid is formed with two clipping grooves.
